(19) United States
(12) Patent Application Publication
(10) Pub. No.: US 2023/0277486 A1
(43) Pub. Date: Sep. 7, 2023

(54) COMPOSITIONS FOR THE TREATMENT OF NON-ARTICULAR CARTILAGE-ASSOCIATED BONE CONDITIONS

(71) Applicant: Lanny Leo Johnson, Henderson, NV (US)

(72) Inventor: Lanny Leo Johnson, Henderson, NV (US)

(21) Appl. No.: 18/316,945

(22) Filed: May 12, 2023

Related U.S. Application Data
(63) Continuation of application No. 16/396,466, filed on Apr. 26, 2019.

(51) Int. Cl. A61K 31/192; A61P 19/10; A61K 35/28; C12N 5/077; A61L 27/38; A61L 27/54; A61K 31/59; A61K 31/122; A61K 33/06

(52) U.S. Cl. CPC A61K 31/192; A61P 19/10; A61K 35/28; C12N 5/0654; A61L 27/3834; A61L 27/54; A61K 31/59; A61K 31/122; A61K 33/06; A61L 2300/43; C12N 2501/999; A61L 2430/02; A61L 2300/21; A61L 2300/414

(57) ABSTRACT

An enhanced bone graft product includes a host matrix, a bone graft material, a scaffold, or a biological construct, and a composition including a therapeutically effective amount of protocatechuic acid. The bone graft material may be autographic, allographic, alloplastic, or xenographic. The bone graft material may include fresh bone, freeze-dried bone, or demineralized freeze-dried bone. The composition may include a pharmaceutically acceptable carrier, adjuvant, or vehicle. The composition may include protocatechuic acid at a concentration of about 10 μM to about 250 μM. The composition may further include mesenchymal stem cells, adipose tissue-derived stem cells, endothelial progenitor cells, mesenchymal skeletal stem cells, synovial stem cells, bone marrow extract, osteoblasts, chondrocytes, osteocytes, or combinations thereof.

### TECHNICAL FIELD

The present invention relates generally to methods for the treatment of various non-articular cartilage related bone conditions, diseases, disorders and or injury; i.e. fractures, delayed unions, non-unions, and metabolic conditions including osteomalacia, osteopenia and osteoporosis. Treatment is achieved by increasing osteogenic activity in bone tissue, which includes administering protocatechuic acid (PCA) directly or indirectly to a bone lesion. PCA may be optionally combined with a bone supplement. This treatment is anabolic in nature and causes osteocytes and or mesenchymal stem cells (MSCs) to produce bone.

### BACKGROUND OF THE INVENTION

Bone tissue is one of the few tissues that retains its regenerative abilities into adult life. Unlike other tissues, bone tissue can heal without forming fibrous scar tissue that results in the bone being restored to a pre-fracture structural and physiological condition. In 10-20% of cases, however, the regenerative process fails, resulting in delayed healing, imperfect unions, or pseudo-arthrosis or non-unions, and other bone conditions such as osteopenia and osteoporosis.

The bone healing process implicates a large number of factors at the molecular level, and their coordinated interaction creates the complex pathways of bone healing. (Giannoudisa P. V., Einhorn T. A., and Marsh D. Fracture Healing: The diamond concept. Injury. 2007 September; 38 Suppl 4:S3-6). The two mechanisms of bone healing are direct (primary) bone healing and indirect (secondary) bone healing. Primary bone healing is rare; it involves intramembranous healing via Haversian remodeling and requires complete stability and minimization of the inter-fragmentary strains. Secondary bone healing is the most common form of bone healing, consisting of both endochondral and intramembranous bone healing, and does not require reduction of interfragmentary strains or rigid stability. Secondary bone healing can be segmented into three stages of healing: 1) the inflammatory phase, 2) the repairing phase, and 3) the bone remodeling phase. Following trauma, the healing process begins with the formation of a fracture hematoma that provides a source of hematopoietic cells capable of secreting growth factors. In response to the injury, macrophages, neutrophils and platelets release proinflammatory cytokines for tissue regeneration. The inflammatory response causes coagulation of the hematoma around the fracture ends and produces a template for callus formation. Osteoclasts are recruited to resorb damaged bone. Mesenchymal stem cells (MSCs) are recruited locally from the surrounding soft tissues, cortex, periosteum, and bone marrow and as well systemically from remote hematopoietic sites via the peripheral blood. These MSCs differentiate into an osteoblastic phenotype, leading to the formation of callus, later replaced by new bone, thereby resulting in bone repair.

There is growing interest in the application of osteoinductive and osteogenic growth factors and mesenchymal stem cells (MSCs) to significantly improve bone repair and regeneration. MSCs are well known in the art for their regenerative abilities. MSCs have been identified in both healthy and diseased tissues. In vitro and in vivo studies have demonstrated their potential in fracture healing, including delayed union and non-union healing. For example, the effectiveness of MSC therapy is dependent on several factors, including the differentiating state of the MSCs at the time of application, their delivery method, the concentration of MSCs per injection, the vehicle used, and the nature and extent of injury. (Oryan, A., Kamali, A., Moshiri, A., and Baghaban Eslaminejad, M. Role of Mesenchymal Stem Cells in Bone Regenerative Medicine. Cells Tissues Organs. 2017; 204(2):59-83). Furthermore, limitations in MSC number and/or functions are hypothesized to play a critical role in fracture healing, including delayed unions and non-unions, and metabolic bone diseases. Thus, MSCs may be utilized to create novel MSC-based therapies that enhance MSCs' osteogenic potential for the treatment of bone disorders and conditions. There is a need in the art, therefore, to provide compositions and techniques that augment MSC-based activities in promoting bone health and healing.

### SUMMARY OF THE INVENTION

The present invention addresses the need to develop compositions and methods for the repair or regeneration of bone. Specifically, the present invention provides anabolic methods and compositions that stimulate or induce bone regeneration and repair, while protecting against degradation of bone. Further, the invention provides methods and compositions that deliver osteogenic activities to the affected region including the enhancement of grafts and scaffolds. Still further, the methods and compositions address the challenges faced with delivery of the desired composition to the affected region. Thus, the present invention provides compositions and methods that may be used to treat a variety of bone conditions, disorders or metabolic diseases.

In one aspect of the present invention, a method of treating a non-articular cartilage-associated bone condition is provided, which includes the administration of a therapeutically effective amount of protocatechuic acid (PCA) to a patient. In some embodiments, the non-articular cartilage-associated bone condition is selected from the group consisting of primary or secondary fractures, surgically created osseous defects, osseous defects created from traumatic injury to bone, progressed traumatic injuries related to failed bone grafting, and other pathologic conditions or metabolic bone diseases. In some embodiments, the pathologic condition or metabolic bone disease is osteoporosis. In some embodiments, the therapeutically effective amount is administered orally, intravenously, subcutaneously, intraperitoneally, or by topical application of PCA crystals to a host matrix, bone graft, scaffold, or biological construct before it is administered to a patient, or directly to a fracture, delayed union, or non-union site. In some embodiments, the therapeutically effective amount administered results in a local concentration of at least 10 μM. In some embodiments, PCA is co-administered with a bone supplement, stems cells, or a combination thereof.

In another aspect of the present invention, a method for promoting osteogenic gene expression in one or more cells is provided, wherein the one or more cells are cultured with a therapeutically effective amount of protocatechuic acid (PCA). In some embodiments, the therapeutically effective amount comprises at least 10 μM. In some embodiments, the one or more cells selected from the group consisting of mesenchymal stem cells (MSCs), adipose tissue-derived stem cells (ADSCs), endothelial progenitor cells (EPCs), mesenchymal skeletal stem cells (SSCs), synovial stem cells, bone marrow extract, osteoblasts, chondrocytes, and osteocytes and combinations thereof. In some embodiments, the one or more cells are derived from autogenic, allogenic or xenogenic sources.

In another aspect of the present invention, a method of enhancing the viability of a bone graft is provided, which includes providing a bone graft; contacting the graft with stem cells; and contacting the bone graft with a therapeutically effective amount of PCA, wherein PCA is applied directly on or in the bone graft. In some embodiments, the stem cells are selected from the group consisting of mesenchymal stem cells (MSCs), adipose tissue-derived stem cells (ADSCs), endothelial progenitor cells (EPCs), mesenchymal skeletal stem cells (SSCs), synovial stem cells, and bone marrow extract and combinations thereof. In some embodiments, the stem cells are derived from autogenic, allogenic or xenogenic sources. In some embodiments, PCA is combined with additional bone growth promoting reagents selected from the group consisting of growth hormone (GH), insulin-like growth factor 1 (IGF-1), parathyroid hormone (PTH), estrogens, calcitonin, calcitriol, osteoprotegerin, leptin, serotonin, and thyroid stimulating hormone (TSH) and combinations thereof. In some embodiments, the therapeutically effective amount administered results in a local concentration of at least 10 μM.

In another aspect of the present invention, a pharmaceutical composition for the treatment of osteoporosis is provided, which includes a therapeutically effective amount of protocatechuic acid (PCA). In some embodiments, the pharmaceutical composition includes a bone supplement selected from the group consisting of calcium, vitamin D, magnesium, and vitamin K and combinations thereof. In some embodiments, the compositions is formulated into a dosage form, the dosage form comprising a tablet, soft capsule, softgel or liquid.

In some embodiments, the therapeutically effective amount administered results in a local concentration of at least 10 μM.

### BRIEF DESCRIPTION OF THE DRAWINGS

FIG. 1 is a chart summarizing the dose dependent effect of PCA on osteogenic gene expression. Osteoblasts were cultured and exposed to PCA at concentrations ranging from 1 μM to 100 μM. The cultures were measured for Bone sialoprotein (IBSP), osteopontin (SPP1), alkaline phosphatase (TNAP) and RunX2 gene expression.

FIG. 2 is a chart summarizing the dose dependent effect of PCA on anti-osteogenic gene expression. Osteoblasts were cultured and exposed to PCA at concentrations ranging from 1 μM to 100 μM. The cultures were measured for TNF-α, IL-6, IL-1bm and MMP-1 gene expression.

FIG. 3 is a chart summarizing the dose dependent effect of PCA on BMP6.

FIG. 4 is a chart summarizing the dose dependent effect of PCA on human mesenchymal stem cells (MSCs) in three cell lines. MSCs were cultured and exposed to PCA for 14 and 21 days at concentrations ranging from 200 μM to 2000 μM (2 mM). Cultured MSCs were measured for SPP1 expression.

FIG. 5 is a chart summarizing the dose dependent effect of PCA on Human mesenchymal stem cells (MSCs) in three cell lines. MSCs were cultured and exposed to PCA for 14 and 21 days at concentrations ranging from 200 μM to 2000 μM (2 mM). Cultured MSCs were measured for RunX expression.

FIG. 6 is a chart summarizing the dose dependent effect of PCA on Human mesenchymal stem cells (MSCs) in three cell lines. MSCs were cultured and exposed to PCA for 14 and 21 days at concentrations ranging from 200 μM to 2000 μM (2 mM). Cultured MSCs were measured for TNAP expression.

FIG. 7 is a chart summarizing the dose dependent effect of PCA on Human mesenchymal stem cells (MSCs) in three cell lines. MSCs were cultured and exposed to PCA for 14 and 21 days at concentrations ranging from 200 μM to 2000 μM (2 mM). Cultured MSCs were measured for IBSP expression.

FIG. 8 depicts the results of Alizarin Red staining at 21 days to test for calcium deposition in three different cell lines treated at concentrations ranging from 200 μM to 2000 μM (2 mM).

FIG. 9 is a chart summarizing the dose dependent effect of PCA on Appl. MSCs relative to control cells.

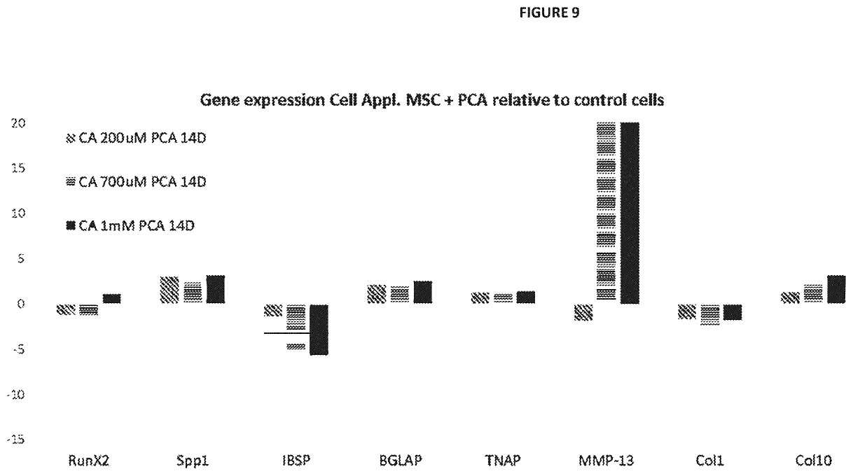

FIG. 10 is a chart summarizing the dose dependent effect of PCA on ATTC MSCs relative to control cells.

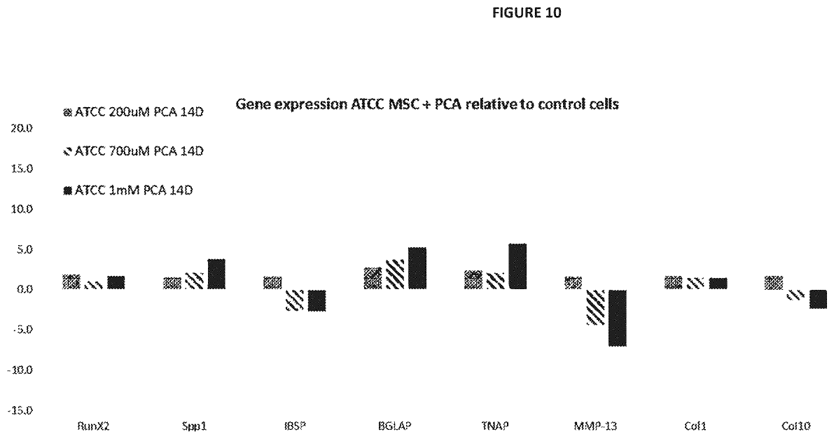

FIG. 11 is a chart summarizing the dose dependent effect of PCA on Rooster MSCs relative to control cells.

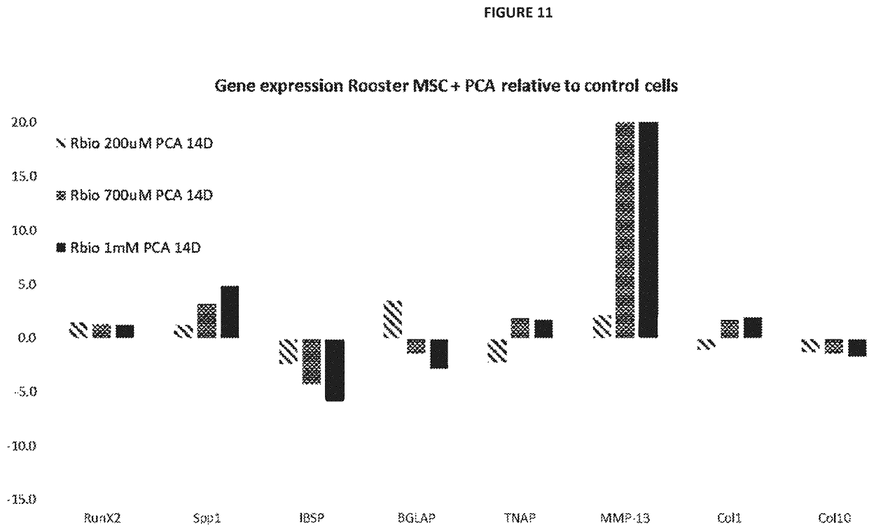

### DETAILED DESCRIPTION OF THE INVENTION

The present invention provides methods, compositions, formulations and uses for treating or preventing various non-articular cartilage-associated bone conditions, disorders or injuries. In particular, the present invention provides methods, compositions and enhanced grafts for the prevention or treatment of non-articular cartilage-associated bone conditions or disorders of the skull, spine, vertebral bodies, clavicle, ribs, sternum, scapula, humerus, ulna, radius, carpals, hand, metacarpals, phalanges, pelvis, femur, patella, tibia, fibula, ankle, talus, foot, cuboids, metatarsals, phalanges, growth plates, intervertebral disc and the like. The present invention is particularly useful as treatment of bone disorders, such as delayed union fractures, non-union fractures, disorders related to failed bone grafting, and metabolic bone diseases like osteopenia and osteoporosis. The present invention provides methods, compositions, formulations and uses to provide for or increase bone formation in bone grafts or on various scaffolds.

The methods and compositions are useful for the treatment of vertebrates. Vertebrates are any animals having a bony or cartilaginous skeleton with a segmented spinal column and a large brain enclosed in a skull or cranium. As such, the methods and treatments are useful for the treatment of amphibians, reptiles, birds, and mammals.

### Definitions

Unless expressly defined otherwise, all technical and scientific terms used herein have the same meaning as is commonly understood by one of ordinary skill in the art to which this invention belongs. All publications referred to throughout the disclosure are incorporated by reference in their entirety. In the event there exists a plurality of definitions or meanings for a term, the interpretation is to be construed consistent with this section and the spirit and scope of this document as a whole.

The terms “active ingredient” or “active pharmaceutical ingredient” as used herein refer to a pharmaceutical agent, active ingredient, compound, or substance, compositions, or mixtures thereof, that provide a pharmacological, often beneficial, effect. In some embodiments, the active ingredient is an anthocyanin or anthocyanidin, or metabolite thereof (e.g., protocatechuic acid, protocatechuic acid ester).

The term “about” as it relates to the resulting concentration after administering a therapeutically effective amount of a phenolic compound means+/−10%. Thus, about 20 μM means 20 μM+/−10%.

The terms “dosage” or “dose” as used herein denote any form of the active ingredient formulation that contains an amount sufficient to produce a therapeutic effect with a single administration.

The term “dosage unit form” as used herein, refers to a physically discrete unit of agent appropriate for the subject to be treated.

As used herein, all percentages (%) refer to weight percent unless noted otherwise.

As used herein, “a” or “an” means one or more unless otherwise specified.

Terms such as “include,” “including,” “contain,” “containing,” “having,” and the like mean “comprising.”

The term “treating” refers to administering a therapy in an amount, manner, or mode effective (e.g., a therapeutic effect) to improve a condition, symptom, disorder, or parameter associated with a disorder, or a likelihood thereof.

The term “patient” or “subject” includes both humans and mammals. The human patient or subject may be a child (˜0-9 years old) adolescent (˜10-17 years old), or an adult (>17 years of age). The patient or subject may be a mammal of any age including dogs, cats, horses, and pigs.

The terms “soft capsule” or “enteric soft capsule” as used herein refer to a soft capsule shell encapsulating a liquid or semisolid “matrix” or “fill” comprising vehicles, pharmaceutically acceptable excipients, and one or more active pharmaceutical ingredients.

The terms “active pharmaceutical ingredient load” or “drug load” as used herein refers to the quantity (mass) of the active pharmaceutical ingredient comprised in a single soft capsule fill.

The terms “formulation” or “composition” as used herein refers to the drug in combination with pharmaceutically acceptable excipients. This term includes orally administrable formulations as well as formulations administrable by other means.

The term “therapeutically effective amount” as used herein refers to an amount of a phenolic compound administered over a sufficient period of time that produces a beneficial orthopedic effect.

The term “proximate,” “proximate to,” or “near to” as used herein refers to a location that is sufficiently near in location that the intended effect or result occurs. For example, PCA proximate to damaged bone is able to affect repair. Injections, application or infusions proximate to damaged bone deliver pharmaceutical composition to damaged bone or bone grafts.

The term “non-articular cartilage associated bone condition” as used herein excludes any bone conditions involving the hyaline or articular cartilage found in the synovial joint.

The term “osteoconductive” refers to the ability of a composition or material to permit bone growth onto and/or into the material.

The term “osteoinductive” refers to the capability of a composition or material to actively stimulate a biological response that induces bone formation. Osteoinduction can include the formation and/or stimulation of osteoprogenitor cells, such as osteoprogenitor cells in bodily tissue surrounding or proximate to a graft or implant.

The term “scaffold” is used to mean one or more layers having properties that can be varied to alter the tissue scaffold's features, for example, strength, volume, porosity and durability, as well as, performance. Scaffold materials include biological or synthetic materials. Scaffold materials include but are not limited to collagen, fibrin, chitosan, hyaluronic acid, carbon nanotube, polyester, polylactic acid, polyglycolic acid, polycaprolactone, coral and printed constructs.

The term “tissue” is used to mean any assemblage of multiple cells and matrix.

### Methods of Treating Non-Articular Cartilage-Associated Bone Conditions and Disorders

In one aspect, the present invention provides a method of treating a non-articular cartilage-associated bone condition comprising administering a therapeutically effective amount of a phenolic compound to a patient.

In one aspect, the present invention provides a method for promoting osteogenic gene expression in one or more cells comprising culturing the one or more cells with a therapeutically effective amount of a phenolic compound.

In one aspect, the present invention provides a method of enhancing the viability of a bone graft comprising: providing a bone graft; contacting the graft with stem cells; and contacting the bone graft with a therapeutically effective amount a phenolic compound. The phenolic compound may be applied directly on or in the bone graft.

In one aspect, the present invention provides a method for promoting growth of a bone tissue comprising administering a therapeutically effective amount of a phenolic compound to the tissue.

In one aspect, the present invention provides a method for priming stem cells to differentiate into a bone tissue comprising culturing the stem cells with a therapeutically effective amount of a phenolic compound.

In some embodiments, the phenolic compound is an anthocyanin, anthocyanidin or a metabolite thereof. In some embodiments, the compound is an anthocyanidin or anthocyanin. Anthocyanidins or anthocyanins for use with the present invention includes those that are isolated, purified from nature, or chemically synthesized. Specifically, anthocyanins and anthocyanidins for use with the present invention are those that are commercially available and provide consistency with pharmacologic standards. Such compounds are well characterized and adapted for pharmaceutical administration. Exemplary and non-limiting anthocyanidins and anthocyanins include cyanidin-3-glucosidase or delphinidin-3-glucosidase, cyanidin-3-galactosidase, and pelargonidin-3-galactosidase, aurantinidin, cyanidin, delphinidin, europinidin, malvidin, pelargonidin, peonidin, petunidin, rosinidin. In some embodiments, the phenolic compound is a metabolite of anthocyanin or anthocyanidin. Exemplary and non-limiting metabolites include: cyanidin-3-glucoside (C3G), protocatechuic acid (PCA), 2,4,6-THBA, phloroglucinaldehyde, vanillic acid, isovanillic acid, VA-4-sulfate, IVA-3-sulfate, ferulic acid, PCA-3-glucuronide, PCA-4-glucuronide, PCA-3-sulfate, PCA-4-sulfate, vanillic acid-glucuronide, isovanillic acid-glucuronide, vanillic acid-sulfate, isovanillic acid-sulfate, methyl 3,4-dihydroxybenzoate, 2-hydroxy-4-methoxybenzoic acid, methyl vanillate, hippuric acid and protocatechuic acid ester.

In some embodiments, the compound is a derivative of PCA. Exemplary and non-limiting derivatives include arieianoic acid, arieianol, and Cyathenosin A.

In some embodiments, the compound is an ester of PCA. Esters can be include C₁-C₈ branched or linear alkyl esters.

In one embodiment, the phenolic compound is PCA.

In some embodiments, the phenolic compound is administered orally, subcutaneously, intramuscularly, intravenously, intrathecally, intraarticularly, juxta-articularly, rectally, sublingually, parenterally, or by topical application of crystals (raw or in a vehicle). The crystals may be applied to a host matrix, bone graft, scaffold, or biological construct before it is administered to a patient, or directly to a fracture, delayed union, or non-union site.

In some embodiments, PCA is administered orally, intravenously, subcutaneously, intraperitoneally, or by topical application of PCA crystals to a host matrix, bone graft, scaffold, or biological construct before it is administered to a patient, or directly to a fracture, delayed union, or non-union site.

In some embodiments, PCA may be administered in a biologically suitable vehicle.

In some embodiments, the phenolic compound administered results in a local concentration of about 1 μM to about 2 mM, about 5 μM to about 1.75 mM, about 10 μM to about 1.5 mM, about 15 μM to about 1.25 mM, about 20 μM to about 1 mM, about 25 μM to about 950 μM, about 30 μM to about 900 μM, about 35 μM to about 850 μM, about 40 μM to about 800 μM, about 45 μM to about 750 μM, about 50 μM to about 700 μM, about 55 μM to about 650 μM, about 60 μM to about 650 μM, about 65 μM to about 600 μM, about 70 μM to about 550 μM, about 75 μM to about 500 μM, about 80 μM to about 450 μM, about 85 μM to about 400 μM, about 90 μM to about 350 μM, about 95 μM to about 300 μM, about 100 μM to about 250 μM, about 105 μM to about 200 μM, and about 110 μM to about 150 μM.

In one embodiment, the PCA administered results in a local concentration of at least 10 μM.

In one embodiment, the phenolic compound administered results in a culture concentration of about 1 μM to about 2 mM, about 5 μM to about 1.75 mM, about 10 μM to about 1.5 mM, about 15 μM to about 1.25 mM, about 20 μM to about 1 mM, about 25 μM to about 950 μM, about 30 μM to about 900 μM, about 35 μM to about 850 μM, about 40 μM to about 800 μM, about 45 μM to about 750 μM, about 50 μM to about 700 μM, about 55 μM to about 650 μM, about 60 μM to about 650 μM, about 65 μM to about 600 μM, about 70 μM to about 550 μM, about 75 μM to about 500 μM, about 80 μM to about 450 μM, about 85 μM to about 400 μM, about 90 μM to about 350 μM, about 95 μM to about 300 μM, about 100 μM to about 250 μM, about 105 μM to about 200 μM, and about 110 μM to about 150 μM.

In one embodiment, the PCA administered results in culture concentration of at least 10 μM.

In some embodiments, the one or more cells are selected from mesenchymal stem cells (MSCs), adipose tissue-derived stem cells (ADSCs), endothelial progenitor cells (EPCs), synovial stem cells, skeletal meneschymal stem cells (SSCs), bone marrow extract, osteoblasts, chondrocytes, or osteocytes and combinations thereof.

In some embodiments, the stem cells are selected from mesenchymal stem cell (MSCs), adipose tissue-derived stem cells (ADSCs), endothelial progenitor cell (EPCs), skeletal mesenchymal stem cell (SSCs), or bone marrow extract and combinations thereof. In one embodiment, the stem cells are MSCs. In some embodiments, the MSCs are Rooster (low osteo), ATTC (medium osteo) or Cell Applications (high osteo).

In some embodiments, stem cells are administered with the phenolic compound. In one embodiment, stem cells are co-administered with PCA. In another embodiment, stem cells are administered before administration of PCA. In another embodiment, stem cells are administered after administration of PCA.

In one embodiment, the non-articular cartilage-associated bone condition is selected from primary or secondary fractures, surgically created osseous defects, osseous defects created from traumatic injury to bone, progressed traumatic injuries related to failed bone grafting, scaffolds, and other pathologic conditions or metabolic bone diseases.

In one embodiment, the pathologic conditions or metabolic bone diseases are selected from osteopenia, osteomalacia, osteoporosis, rickets, chemotherapy-induced bone loss, post-menopausal bone loss, parathyroid disorders, calcium and phosphorus disorders, renal osteodystrophy, pathologic fractures, and Paget's disease.

In one embodiment, the pathologic condition or metabolic bone disease is osteoporosis. Cases of osteoporosis are frequently found in astronauts. Therefore, the invention anticipates treatment for osteoporosis resulting from prolonged exposure to microgravity.

In one embodiment, the bone condition is a primary or secondary fracture. Secondary fractures are those fractures that occur due to pathological weakening of the bone stemming from osteoporosis, osteolysis, metastasis, infection, metabolic disorders, or other systemic or local disease. In some embodiments, the primary or secondary fracture is a traverse fracture, oblique fracture, comminuted fracture, Greenstick fracture, stress fracture, spiral fracture, compression fracture, segmental loss fracture, displaced fracture, overriding fracture, angulated fracture, mal-union fracture or delayed union fracture.

In some embodiments, the phenolic compound promotes osteogenic gene expression in the one or more cells. In one embodiment, PCA promotes osteogenic expression in one or more cells. In one embodiment, PCA promotes osteogenic gene expressions in MSCs. In one embodiment, PCA promotes osteogenic gene expression in osteoblasts. In some embodiments, the phenolic compound upregulates one or more genes selected from osteopontin, osteoprotegerin, RUNX2, osterix, Dlx5, Msx-2, c-fos, Cox-2, FGF2, Bapx1, osteocalcin, β-catenin, ATF4, SatB2, Collagen I, alkaline phosphatase, bone sialoprotein (BSP), or BMP-6. In some embodiments, the phenolic compound downregulates expression of one or more genes selected from TNF-α, MMP-1, NF-κB, JNK, Twist1, IL-1β and IL-6. In one embodiment, PCA upregulates one or more genes selected from osteopontin, RUNX2, alkaline phosphatase, BSP, or BMP-6. In one embodiment, PCA downregulates one or more genes selected from TNF-α, LMX1B, MMP-1. In some embodiments, the phenolic compound increases calcium deposition in one or more cells. In one embodiment, PCA increases calcium deposition in MSCs. In some embodiments, bone growth is promoted around an implant or internal fixation device.

In some embodiments, the phenolic compound is administered with a bone supplement selected from calcium, vitamin D, magnesium, vitamin K and combinations thereof. In some embodiments, calcium is selected from calcium carbonate, calcium phosphate, calcium hydroxide, calcium chloride, calcium sulfate, calcium citrate, microcrystalline hydroxyapatite calcium (MCHC), calcium citrate malate, calcium lactate, calcium ascorbate, calcium gluconate, calcium orotate, calcium acetate, and lithothamnion superpositum. In some embodiments, vitamin D is selected from vitamin D2 and vitamin D3. In some embodiments, magnesium is selected from magnesium threonate, magnesium glycinate, magnesium malate, magnesium oxide, magnesium orotate, magnesium chloride, magnesium sulfate, magnesium taurate, magnesium citrate, magnesium hydroxide, and magnesium aspartate and glutamate. In some embodiments, vitamin K is selected from phylloquinones, menaquinone-4 and menaquinone-7.

In some embodiments, the phenolic compound is combined with additional bone growth promoting reagents selected from growth hormone (GH), insulin-like growth factor 1 (1GF-1), parathyroid hormone (PTH), estrogens, calcitonin, calcitriol, osteoprotegerin, leptin, serotonin, or thyroid stimulating hormone (TSH) and combinations thereof. In one embodiment, PCA is combined with additional bone growth promoting reagents selected from growth hormone (GH), insulin-like growth factor 1 (IGF-1), parathyroid hormone (PTH), estrogens, calcitonin, calcitriol, osteoprotegerin, leptin, serotonin, or thyroid stimulating hormone (TSH) and combinations thereof.

### Enhanced Bone Grafts and Scaffolds

One aspect of the invention provides an enhanced bone graft composition comprising a therapeutically effective amount of a phenolic compound, stem cells, and a bone grafting material.

In one embodiment, the phenolic compound is PCA.

In one embodiment, the therapeutically effective amount of PCA may be applied directly to the bone graft in the form of crystals and or in a suitable biological vehicle.

One aspect of the invention provides an enhanced bone scaffold comprising stem cells and a therapeutically effective amount of a phenolic compound.

A bone graft composition (or material) or scaffold according to some embodiments is configured to facilitate repair, replacement, or regeneration of bone at a target repair site. In some embodiments, the target repair site can be, for example, a void, gap, or other defect in a bone or other bony structure in a body of a patient. In some embodiments, the bone graft composition or scaffold is configured to be implanted or otherwise disposed at the target repair site. In some embodiments, the graft results in enhanced osteoconductive, osteoinducive, or osteogenic benefit, or a combination thereof.

In some embodiments, the bone graft material may be autographic, allographic, alloplastic, or xenographic. In some embodiments, the autograph may be harvested from non-essential bones, such as the iliac crest, fibula, or ribs. In some embodiments, the autograft may be performed without a solid bony structure. In some embodiments, the allograft may be fresh bone, freeze-dried or demineralized freeze-dried bone allograft. In some embodiments, the alloplastic grafts may be made from various materials including collagens, bioactive glasses, calcium phosphates, calcium sulphate and polylactic and polyglycolic acids, and combinations thereof.

In some embodiments, the scaffold is made of biological components. In some embodiments, the scaffold can be made of collagen, fibrin, chitosan, hyaluronic acid and combinations thereof. In other embodiments, the scaffold is made of synthetic components such as carbon nanotube or linear aliphatic polyester. In other embodiments, the scaffold is made of polylactic acid, polyglycolic acid, polycaprolactone, and combinations thereof. The scaffold can be any suitable shape. In general, the suitable shape is any shape compatible for the particular bone structure being remodeled as will be appreciated by the skilled person, e.g., planar, tubular, cylindrical, conical, truncated cone, a pentahedron, a truncated-pentahedron, an elliptical or oval shaped column, or a combination thereof.

In some embodiments, the scaffolds are 3-D printed. 3D printing techniques may include, but are not limited to, extrusion-based and inkjet-based 3D printing methods. In the inkjet printing method, biological materials are selectively placed onto the build platform in a layer-by-layer manner until the required construct is formed. In extrusion-based bioprinting, biomaterials are extruded from the print-head due to the exertion of mechanical or pneumatic pressure. 3-D printing techniques may also include fiber bonding, phase separation, solvent casting, particulate leaching, membrane lamination, molding, foaming, fused deposition modeling (FDM), stereolithography, selective laser sintering (SLS), and colorjet printing.

In some embodiments, materials suitable for 3D-printing processes include solidifiable fluid, non-brittle filament, laminated thin sheet, and fine powder. Solidifiable fluid includes photopolymer resins, temperature sensitive polymers, ion cross-linkable hydrogels, ceramic paste and the like. Non-brittle filament includes thermoplastics such as ABS, PLA, and PCL. Laminated thin sheet includes paper, plastic sheet, and metal foil. Fine powders includes plastic fine powder, ceramic powder, and metal powder.

In some embodiments, materials used for 3D-printing processes include nanocellulose, alginate, collagen, gelatin, alginate hydrogel, gellan, gamma-irradiated alginate, poly(ε-caprolactone) (PCL) fibers, cartilage extracellular matrix particles, M13 phages, human adipose stem cells (hASCs), carboxymethyl-chitosan, agarose, commercial polyethylene glycol (PEG)-based bioink, gelatin-based bioink, poly(ethylene glycol) diacrylate (PEGDA), gelatin methacrylate (GelMA), eosin Y based photoinitiator, PCL/alginate mesh, methacrylated gelatin (GM), mature adipocytes, hyaluronic acid polyurethane (PU), c2c12 cells, NIH/3T3 cells, hyaluronic acid, gelatin, fibrinogen, PCL olyethylene oxide (PEO), HEK293 cells, human umbilical vein endothelial cells (HUVECs), acrylated, pluronic F127, alginate in phosphate-buffered saline (PBS), hASCs, type I collagen and chitosan-agarose blends, human bone marrow derived mesenchymal stem cells (hMSCs), decellularized adipose tissue (DAT) matrix bioink, Spider silk protein, human fibroblasts, poly(N-isopropylacrylamide), poly(N-isopropylacrylamide), grafted hyaluronan (HA-pNIPAAM), methacrylated hyaluronan (HAMA), nanofibrillated cellulose (NFC), decellularized adipose (adECM), cartilage (cdECM), and heart (hdECM) tissue, and combinations thereof.

In some embodiments, the phenolic compound is an anthocyanin, anthocyanidin or a metabolite thereof. In some embodiments, the phenolic compound is PCA.

In some embodiments, the stem cells are selected from mesenchymal stem cell (MSCs), adipose tissue-derived stem cells (ADSCs), endothelial progenitor cell (EPCs), skeletal mesenchymal stem cell (SSCs), or bone marrow extract and combinations thereof. In one embodiment, the stem cells are MSCs. In some embodiments, the MSCs are Rooster (low osteo), ATTC (medium osteo) or Cell Applications (high osteo).

In one embodiment, PCA is combined with additional bone growth promoting reagents selected from growth hormone (GH), insulin-like growth factor 1 (IGF-1), parathyroid hormone (PTH), estrogens, calcitonin, calcitriol, osteoprotegerin, leptin, serotonin, thyroid stimulating hormone (TSH), and combinations thereof.

### Pharmaceutical Compositions, Dosages, Dosage Forms and Routes of Administration

One aspect described herein is a pharmaceutical composition for the treatment of a non-articular cartilage-associated bone condition comprising a formulation of a phenolic compound.

In one embodiment, the phenolic compound is PCA.

In one embodiment, the pharmaceutical composition further comprises a bone supplement selected from calcium, vitamin D, magnesium, and vitamin K, and combinations thereof.

In one embodiment, the pharmaceutical composition is formulated into a dosage form, the dosage form comprising a tablet, soft capsule, softgel or liquid.

In one embodiment, the non-articular cartilage-associated bone condition is selected from one or more of: primary or secondary fractures, surgically created osseous defects, osseous defects created from traumatic injury to bone, progressed traumatic injuries related to failed bone grafting, and other pathologic conditions or metabolic bone diseases.

In one embodiment, the pathologic conditions or metabolic bone diseases are selected from osteopenia, osteomalacia, osteoporosis, rickets, chemotherapy-induced bone loss, post-menopausal bone loss, parathyroid disorders, calcium and phosphorus disorders, renal osteodystrophy, pathologic fractures, and Paget's disease.

In one embodiment, the pathologic condition or metabolic disease is osteoporosis.

Pharmaceutical Compositions

In the methods described and claimed herein, the phenolic compound and a bone supplement may be administered in the form of a pharmaceutical composition comprising the phenolic compound, a bone supplement and a pharmaceutically acceptable carrier, adjuvant or vehicle.

The term “pharmaceutically acceptable carrier, adjuvant, or vehicle” includes any and all solvents, diluents, or other liquid vehicles, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the phenolic compound, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Dosages

The phenolic compound may be administered in any amount appropriate to treat a non-articular cartilage-associated bone condition or disorder. In some embodiments, a therapeutically effective amount of a phenolic compound is administered daily to achieve a local concentration of about 1 μM to about 2 Mm, about 5 μM to about 1.75 mM, about 10 μM to about 1.5 mM, about 15 μM to about 1.25 mM, about 20 μM to about 1 mM, about 25 μM to about 950 μM, about 30 μM to about 900 μM, about 35 μM to about 850 μM, about 40 μM to about 800 μM, about 45 μM to about 750 μM, about 50 μM to about 700 μM, about 55 μM to about 650 μM, about 60 μM to about 650 μM, about 65 μM to about 600 μM, about 70 μM to about 550 μM, about 75 μM to about 500 μM, about 80 μM to about 450 μM, about 85 μM to about 400 μM, about 90 μM to about 350 μM, about 95 μM to about 300 μM, about 100 μM to about 250 μM, about 105 μM to about 200 μM, and about 110 μM to about 150 μM. In one embodiment, a therapeutically effective amount of PCA is administered daily to achieve a local concentration of at least 10 μM.

The phenolic compound, when administered for multiple days, may be administered in the same or different concentrations each day. In some embodiments, the phenolic compound is administered in the same amount each day. In some aspects, the phenolic compound is administered in an amount to achieve different local concentrations on the first day and after the first day.

The phenolic compound may be administered in any number of doses per day. In some embodiments, the phenolic compound is administered in two or more doses per day. In some embodiments, the phenolic compound is administered in the same number of doses on the first day and after the first day. In some embodiments, the phenolic compound is administered in two doses on the first day (i.e., a first dose and a subsequent dose). The quantity of the first dose and the subsequent dose may be the same or different. In some embodiments, the first dose is larger than the subsequent dose on the first day.

Dosage Forms and Routes of Administration

The methods described and claimed herein may involve the administration of the phenolic compound by any route of administration effective for treating one or more non-articular cartilage-associated bone conditions or disorders. The phenolic compound may be formulated in dosage unit form for ease of administration and uniformity of dosage.

The phenolic compound, derivative or ester thereof, can be administered to humans and other vertebrate orally, rectally, parenterally, intravenously, intracisternally, intraperitoneally, topically (as by powders, ointments, drops, or crystals), bucally, directly on the treatment site, as an oral or nasal spray, or the like, depending on the condition being treated.

Liquid dosage forms for oral administration include, but are not limited to, pharmaceutically acceptable emulsions, microemulsions, solutions, suspensions, syrups and elixirs. In addition to the phenolic compound, derivative or ester thereof, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, or slurries may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Gamma irradiation or the like may be used.

In order to prolong the effect of the phenolic compound, it may be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide, amorphous 50/50 D,L lactide glycolide or 85/15 D,L lactide glycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, gelcaps, pills, powders, and granules. In such solid dosage forms, the phenolic compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. The dosage form may comprise absorbance enhancers, including quercetin, genistein, naringin, sinomenine, piperine, glycyrrhizin, cyclodextrin and nitrile glycoside.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compound also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the active compound or salt may be admixed with at least one inert diluent such as sucrose, lactose or starch. Such dosage forms may also comprise additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of the phenolic compound include ointments, pastes, creams, lotions, gels, powders, solutions, sprays, inhalants, patches or crystals. The active component is admixed under sterile conditions with a pharmaceutically acceptable carrier and any needed preservatives or buffers as may be required.

In one embodiment, the composition is provided as a solution for injection or infusion or one that can be suspended such that it can be injected or infused at the site requiring treatment. In another embodiment, the composition is provided in tablet form. In another embodiment, the composition is provided in capsule form.

### EXAMPLES

### Example 1: Assessment of Osteogenic Effect of Protocatechuic Acid

Protocatechuic acid (PCA) increased osteogenic gene expression in a dose-dependent manner. PCA at the highest concentration tested (100 μM) increased gene expression even greater than osteogenic media. PCA also increased expression of the growth factor BMP-6. PCA had a mixed effect on anti-osteogenic genes: reducing levels of MMP-1 and TNF-α, while increasing IL-1 and IL-6. PCA had no detectable effect on calcium deposition. PCA did not increase deposition of calcium by detectable amounts. The concentration of calcium was higher in osteogenic media (0.20 g/L) than for the control media (0.15 g/L), and this could be a factor affecting calcium deposition. Another potential reason for lack of calcium deposition is that 12 days of culture was not long enough for PCA to induce calcium deposition.

### Materials and Methods

Cells: Primary human osteoblasts were obtained from Cell Applications, San Diego, Calif.

Cell culture: Osteoblasts were expanded in culture using the protocol provided by Cell Applications. Human Osteoblast Basal Media was supplemented with Human Osteoblast Growth Supplement (Cell Applications) containing 0.15 g/L calcium. Human Osteoblast Differentiation Media was purchased from Cell Applications, containing 0.20 g/L calcium.

Treatment: Osteoblasts were cultured in in Human Osteoblast Basal Media and exposed to Protocatechuic acid (PCA) for up to 12 days at concentrations ranging from 1 μmolar to 100 μM. Osteoblasts were cultured without any PCA as negative control. Osteoblasts were cultured without any PCA in osteogenic media (Human Osteoblast Differentiation Media) as positive control (osteogenic media induces osteogenesis in osteoblasts). Each experiment was conducted in triplicate. Media were changed every 3 days.

Gene expression: Pro-osteogenic and anti-osteogenic gene expression was measured using qPCR on mRNA extracted from osteoblast cultures. Gene expression was computed as fold-change relative to control group (osteoblasts cultured without any PCA).

Calcium deposition: Osteoblast cultures were tested for calcium deposition using Alizarin red stain.

### Results

### Anti-Osteogenic Gene Expression

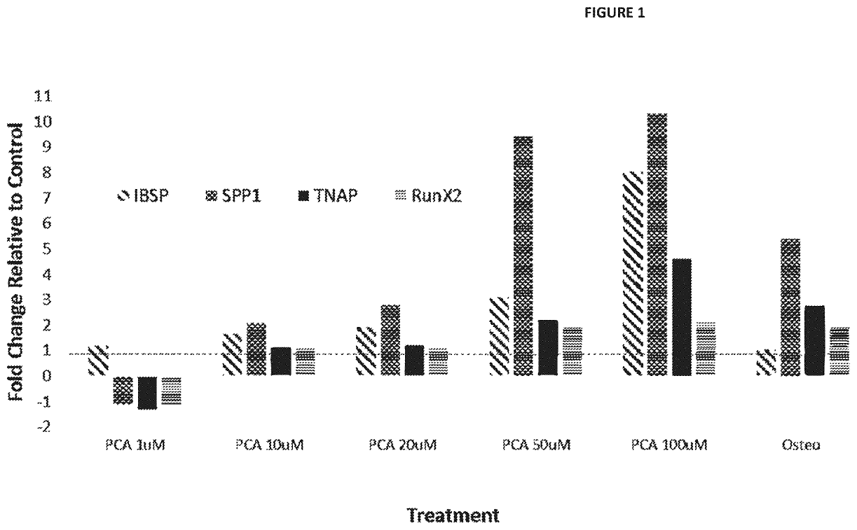

PCA had a dose-dependent effect on all osteogenic genes tested (FIG. 1). The largest effects were seen at the highest dose (100 μmolar) on the expression of IBSP and SPP1. At this dose, the expression of osteogenic genes was even higher than that measured in the positive control (osteoblasts cultured without any PCA in osteogenic media).

### Anti-Osteogenic Gene Expression

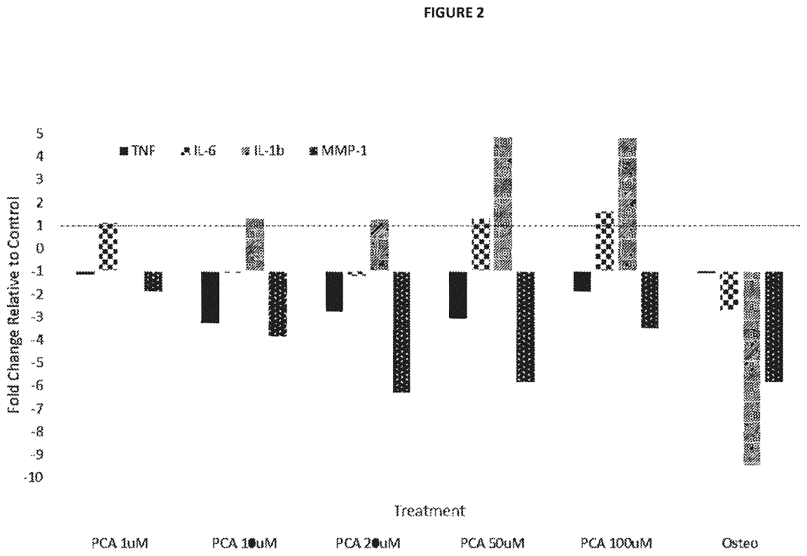

PCA had a mixed effect on anti-osteogenic genes, suppressing TNF-α and MMP-1, but increasing IL-1β and IL-6 (see FIG. 2).

### Growth Factor Gene Expression

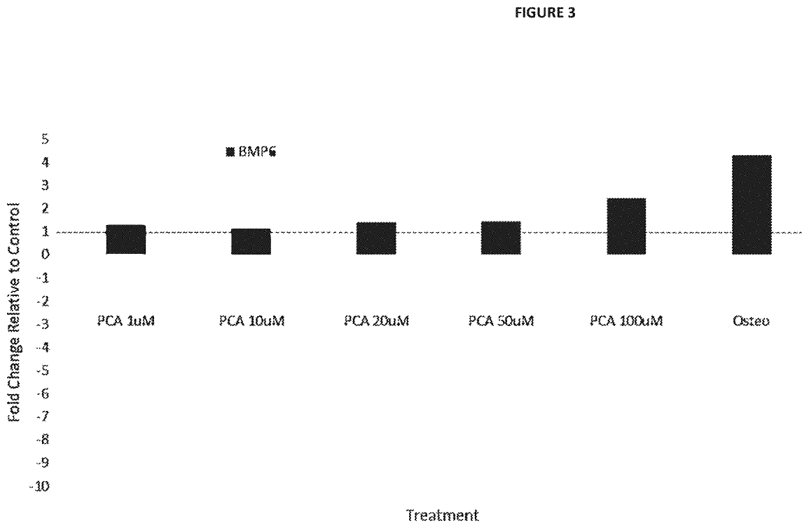

PCA also had a dose-dependent effect on BMP-6 expression. At 100μmolar, BMP-6 rose greater than 2-fold relative to negative control (FIG. 3).

### Calcium Deposition

PCA did not increase deposition of calcium by detectable amounts. The positive control (osteoblasts cultured in osteogenic media) did show evidence of calcium deposition. The concentration of calcium was higher in osteogenic media (0.20 g/L) than for the control media (0.15 g/L), and this could be a factor affecting calcium deposition. Another potential reason for lack of calcium deposition is that 12 days of culture was not long enough for PCA to induce calcium deposition.

### Example 2: Assessment of Osteogenic Effect of Protocatechuic Acid on Human Mesenchymal Stem Cells

Protocatechuic acid (PCA) increased expression of important osteogenic genes in mesenchymal stem cells. PCA increased Osteopontin and RUNX2 expression even greater than the osteogenic conditions. PCA increased expression of alkaline phosphatase compared to control but not as much as osteogenic conditions. PCA induced deposition of calcium in at least one cell line by 3 weeks.

### Materials and Methods

Cells: Primary human mesenchymal stem cells (MSC) were obtained from RoosterBio Inc., Frederick, Md.; ATCC, Manassas, Va.; and Cell Applications, San Diego, Calif.

Cell culture: MSC were expanded in culture, using the protocol provided by the respective suppliers, before being subjected to the experimental conditions.

Treatment: MSC were cultured in supplier-provided culture media and exposed to Protocatechuic acid (PCA) for 14 or 21 days at concentrations ranging from 200 μM to 2000 μM (2 mM). MSC were cultured without any PCA as negative control. To serve as positive control, MSC were cultured without any PCA in osteogenic media (Human Osteoblast Differentiation Media, Cell Applications, Catalog number: 417D-250). Osteogenic media is commonly used to induce osteogenesis. Each experiment was conducted in triplicate. Media was changed twice a week.

Gene expression: Osteogenic gene expression was measured using qPCR on mRNA extracted from MSC cultures. Gene expression was computed as fold-change relative to the control group (MSC cultured without any PCA). MSC cultured in osteogenic media was used as positive control as this protocol is commonly used to induce osteogenesis.

Calcium deposition: MSC cultures were tested for calcium deposition using Alizarin red stain at 21 days.

### Results

### Osteogenic Gene Expression

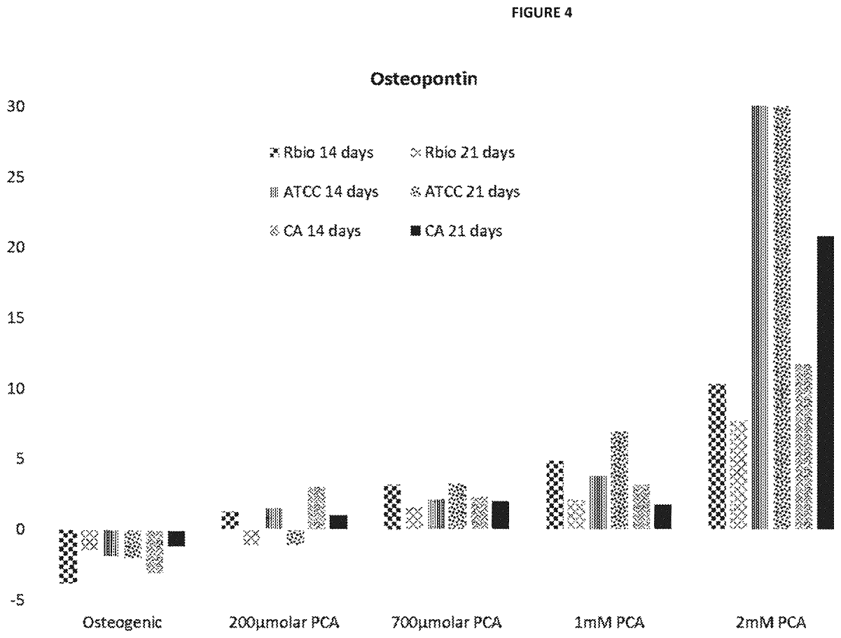

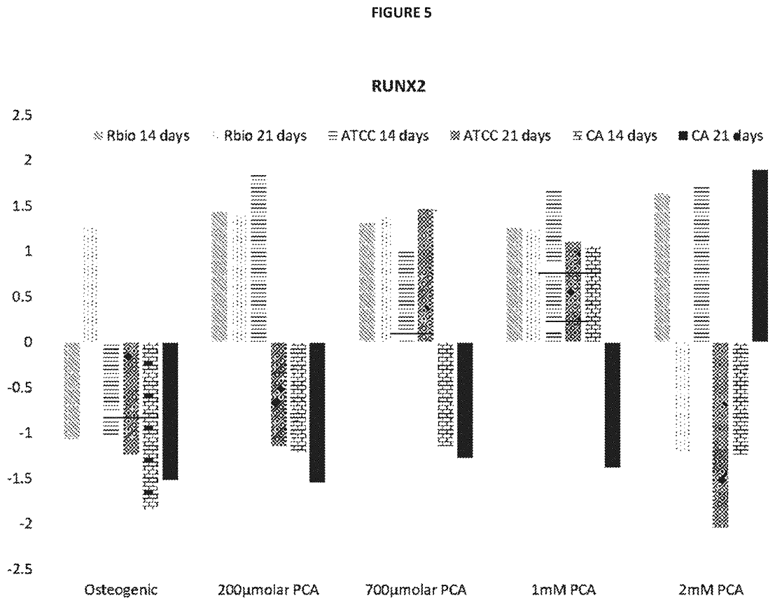

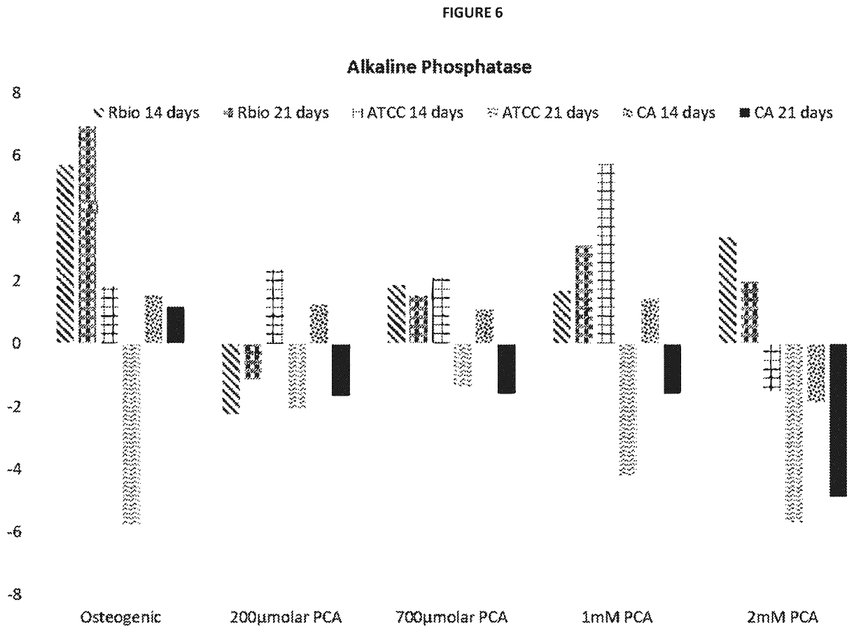

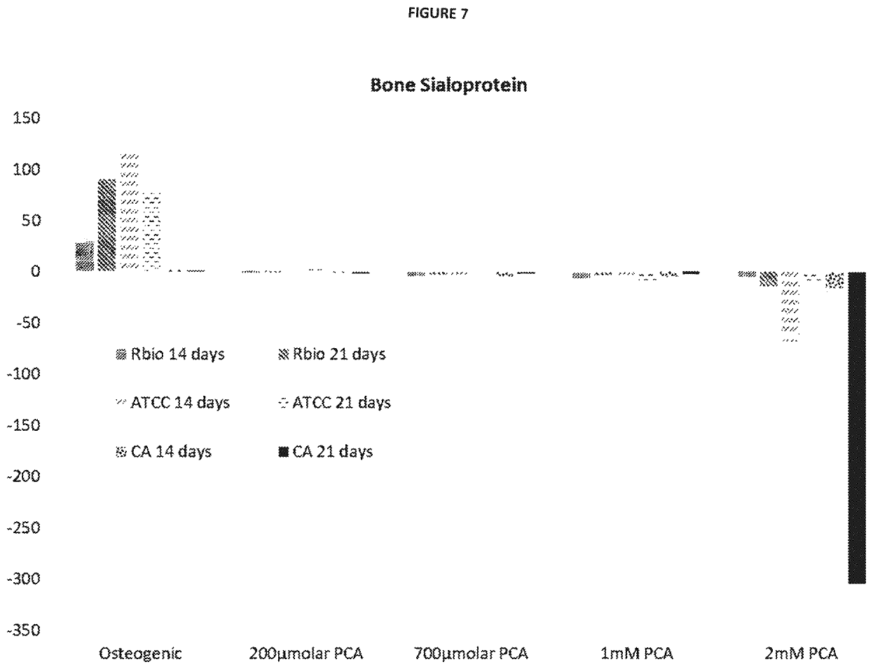

PCA had positive effect on three of the four osteogenic genes tested (FIGS. 4-7). The largest effect was noted in the expression of osteopontin (FIG. 4), which plays a critical role in the bone repair and maintenance. At all doses and in all three cell lines, the expression of osteopontin was even higher than that measured in the positive control (MSC cultured without any PCA in osteogenic media).

PCA also increased RUNX2 at most dose levels. Overall RUNX2 expression was higher at 2 weeks than at 3 weeks. RUNX2 expression was mostly higher than the positive control.

PCA increased the expression of alkaline phosphatase relative to negative control but not greater than the osteogenic conditions.

PCA did not increase the expression of bone sialoprotein relative to either negative or positive controls.

The above results were broadly similar in all three MSC lines.

### Calcium Deposition

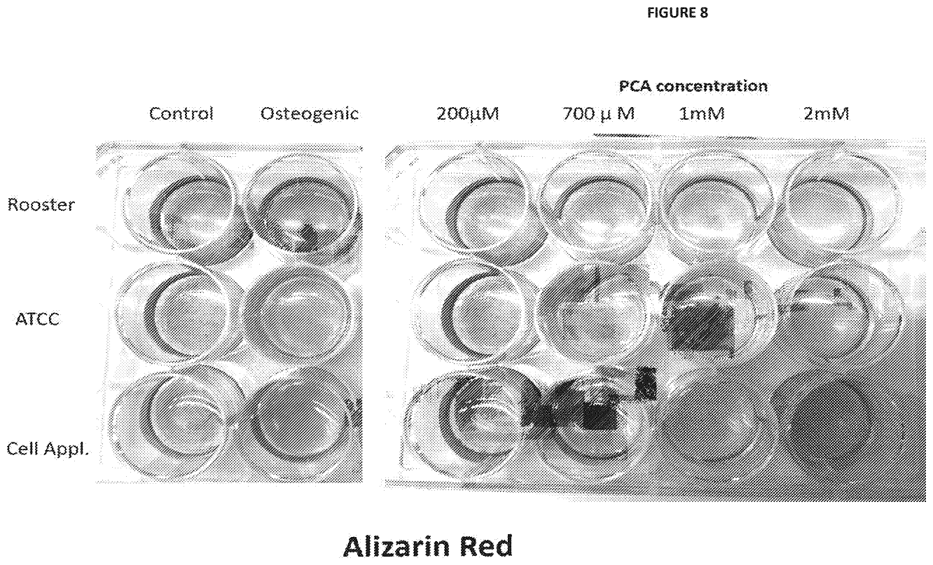

PCA visibly increased the deposition of calcium in one of the three cell lines (FIG. 8). Possible reasons for lack of deposition in other cells lines could be donor-to-donor variability or that the effect on calcium deposition, which occurs later in the osteogenic timeline, could be more apparent after 3 weeks.

### Example 3: Screening PCA for Osteogenesis of hMSC of Low, Medium and High Osteogenic Capacity

Following the screening of three commercial sources of human MSC, we have identified cell sources with low, medium and high osteogenic and calcium deposition capacity. This range of response is convenient to determine whether PCA cannot only enhance osteogenesis, but may boost low to medium capacity donors.

### Materials and Methods

Cells: Rooster (low osteo), ATCC (medium osteo), Cell Applications (high osteo).

Conditions: expansion medium (negative), osteogenic medium (positive), PCA at four concentrations: 200 μM, 700 μM, 1 mM, and 2 mM.

Time Points: 14 days and 21 days

Outcome measures: gene expression (14 and 21 days). Alizarin red and von Kassa (21 days)

Plate 1 PCA Treatments (14 Day RNA)

| | | | |
|---|---|---|---|
| Ro 200 uM PCA | Ro 700 uM PCA | Ro 1 mM PCA | Ro 2 mM PCA |
| ATCC 200 uM | ATCC 700 uM | ATCC 1 mM | ATCC 2 mM PA |
| PCA | PCA | PCA | PCA |
| Cap 200 uM PCA | Cap 700 uM PCA | Cap 1 mM PCA | Cap 2 mM PCA |

Plate 2 PCA Treatments (21 Day RNA)

| | | | |
|---|---|---|---|
| Ro 200 uM PCA | Ro 700 uM PCA | Ro 1 mM PCA | Ro 2 mM PCA |
| ATCC 200 uM | ATCC 700 uM | ATCC 1 mM | ATCC 2 mM |
| PCA | PCA | PCA | PCA |
| Cap 200 uM PCA | Cap 700 uM PCA | Cap 1 mM PCA | Cap 2 mM PCA |

Plate 3 PCA Treatments (21 Day Alizarin Red)

| | | | |
|---|---|---|---|
| Ro 200 uM PCA | Ro 700 uM PCA | Ro 1 mM PCA | Ro 2 mM PCA |
| ATCC 200 uM | ATCC 700 uM | ATCC 1 mM | ATCC 2 mM |
| PCA | PCA | PCA | PCA |
| Cap 200 uM PCA | Cap 700 uM PCA | Cap 1 mM PCA | Cap 2 mM PCA |

Plate 4 Positive, Negative Controls and Combined PCA Treatment (14 Day RNA)

| | | | |
|---|---|---|---|
| Ro Control | Ro Osteo only | Ro Osteo + 700 uM | Ro Osteo + 1 mM |
| | | PCA | PCA |
| ATCC Control | ATCC Osteo | ATCC + 700 uM | ATCC + 1 mM |
| | only | PCA | PCA |
| Cap Control | Cap Osteo only | Cap + 700 uM PCA | Cap + 1 mM PCA |

Plate 5 Positive, Negative Controls and Combined PCA Treatment (21 Day RNA)

| | | | |
|---|---|---|---|
| Ro Control | Ro Osteo only | Ro Osteo + 700 uM | Ro Osteo + 1 mM |
| | | PCA | PCA |
| ATCC Control | ATCC Osteo | ATCC + 700 uM | ATCC + 1 mM |
| | only | PCA | PCA |
| Cap Control | Cap Osteo only | Cap + 700 uM PCA | Cap + 1 mM PCA |

Plate 6 Positive, Negative Controls and Combined PCA Treatment (21 Day Alizarin Red)

| | | | |
|---|---|---|---|
| Ro Control | Ro Osteo only | Ro Osteo + 700 uM | Ro Osteo + 1 mM |
| | | PCA | PCA |
| ATCC Control | ATCC Osteo | ATCC + 700 uM | ATCC + 1 mM |
| | only | PCA | PCA |
| Cap Control | Cap Osteo only | Cap + 700 uM PCA | Cap + 1 mM PCA |

Plate 7 3 PCA Treatments (21 Day Von Kossa)

| | | | |
|---|---|---|---|
| Ro 200 uM PCA | Ro 700 uM PCA | Ro 1 mM PCA | Ro 2 mM PCA |
| ATCC 200 uM | ATCC 700 uM | ATCC 1 mM | ATCC 2 mM |
| PCA | PCA | PCA | PCA |
| Cap 200 uM PCA | Cap 700 uM PCA | Cap 1 mM PCA | Cap 2 mM PCA |

Plate 8 Positive, Negative Controls and Combined PCA Treatment (21 Day Von Kassa)

| | | | |
|---|---|---|---|
| Ro Control | Ro Osteo only | Ro Osteo + 700 uM | Ro Osteo + 1 mM |
| | | PCA | PCA |
| ATCC Control | ATCC Osteo | ATCC + 700 uM | ATCC + 1 mM |
| | only | PCA | PCA |
| Cap Control | Cap Osteo only | Cap + 700 uM PCA | Cap + 1 mM PCA |

1. An enhanced bone graft product comprising:
a host matrix, a bone graft material, a scaffold, or a biological construct; and
a composition comprising a therapeutically effective amount of protocatechuic acid.

2. The enhanced bone graft product of claim 1, wherein the bone graft product comprises the bone graft material and the bone graft material is autographic, allographic, alloplastic, or xenographic.

3. The enhanced bone graft product of claim 1, wherein the bone graft product comprises the bone graft material and the bone graft material is harvested from an iliac crest bone, a fibula bone, or a rib bone.

4. The enhanced bone graft product of claim 1, wherein the bone graft product comprises the bone graft material and the bone graft material comprises fresh bone, freeze-dried bone, or demineralized freeze-dried bone.

5. The enhanced bone graft product of claim 1, wherein the bone graft product comprises the bone graft material and the bone graft material comprises collagens, bioactive glasses, calcium phosphates, calcium sulphate, polylactic and polyglycolic acids, or combinations thereof.

6. The enhanced bone graft product of claim 1, wherein the bone graft product comprises the scaffold and the scaffold comprises collagen, fibrin, chitosan, hyaluronic acid, carbon nanotube, linear aliphatic polyester, polylactic acid, polyglycolic acid, polycaprolactone, or combinations thereof.

7. The enhanced bone graft product of claim 1, wherein the bone graft product comprises the scaffold and the scaffold is 3-D printed.

8. The enhanced bone graft product of claim 7, wherein the 3-D printing comprises extrusion-based or inkjet-based 3D printing.

9. The enhanced bone graft product of claim 1, wherein the bone graft product comprises a host matrix and a bone graft material.

10. The enhanced bone graft product of claim 1, wherein the composition comprises growth hormone (GH), insulin-like growth factor 1, parathyroid hormone, estrogens, calcitonin, calcitriol, osteoprotegerin, leptin, serotonin, thyroid stimulating hormone (TSH), or combinations thereof.

11. The enhanced bone graft product of claim 1, wherein the composition comprises calcium, vitamin D, magnesium, vitamin K, or combinations thereof.

12. The enhanced bone graft product of claim 11, wherein the calcium comprises calcium carbonate, calcium phosphate, calcium hydroxide, calcium chloride, calcium sulfate, calcium citrate, microcrystalline hydroxyapatite calcium, calcium citrate malate, calcium lactate, calcium ascorbate, calcium gluconate, calcium orotate, calcium acetate, or lithothamnion superpositum.

13. The enhanced bone graft product of claim 11, wherein the vitamin D comprises vitamin D2 or vitamin D3.

14. The enhanced bone graft product of claim 11, wherein the magnesium comprises magnesium threonate, magnesium glycinate, magnesium malate, magnesium oxide, magnesium orotate, magnesium chloride, magnesium sulfate, magnesium laurate, magnesium citrate, magnesium hydroxide, magnesium aspartate, or magnesium glutamate.

15. The enhanced bone graft product of claim 11, wherein the vitamin K comprises phylloquinones, menaquinone-4, or menaquinone-7.

16. The enhanced bone graft product of claim 1, wherein the composition comprises a pharmaceutically acceptable carrier, adjuvant, or vehicle.

17. The enhanced bone graft product of claim 1, wherein the composition comprises protocatechuic acid at a concentration of about 10 μM to about 250 μM.

18. The enhanced bone graft product of claim 17, wherein the composition comprises protocatechuic acid at a concentration of about 75 μM to about 250 μM

19. The enhanced bone graft product of claim 1, wherein the composition further comprises mesenchymal stem cells, adipose tissue-derived stem cells, endothelial progenitor cells, mesenchymal skeletal stem cells, synovial stem cells, bone marrow extract, osteoblasts, chondrocytes, osteocytes, or combinations thereof.
